(19) United States
(12) Patent Application Publication
(10) Pub. No.: US 2004/0088798 A1
(43) Pub. Date: May 13, 2004

(54) Oxidation dyeing composition for keratinous fibres and dyeing method using same

(76) Inventor: Gerard Lang, Saint Prix (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/363,147

(22) PCT Filed: Mar. 5, 2001
(86) PCT No.: PCT/FR01/00645

(30) Foreign Application Priority Data
Mar. 6, 2000 (FR) 00/02861

(51) Int. Cl. A61K 7/13

(52) U.S. Cl. 8/405

(57) ABSTRACT

The invention concerns an oxidation dyeing composition for keratinous fibres, and in particular human keratinous fibres such as hair comprising, in a suitable dyeing medium, at least an oxidation base selected among certain substituted para-phenylenediamine derivatives and their addition salts with an acid, at least a polymer selected among amphoteric polymers, cationic polymers with specific repeat still units, or amphiphilic polymers comprising at least a fatty chain, and the dyeing method using said composition.

The invention relates to a composition for the oxidation dyeing of keratin fibers, and in particular of human keratin fibers such as the hair, comprising, in a medium that is suitable for dyeing, at least one oxidation base chosen from certain substituted para-phenylenediamine derivatives and the addition salts thereof with an acid and at least one specific polymer, and also to the dyeing process using this composition.

It is known practice to dye keratin fibers, and in particular human hair, with dye compositions containing oxidation dye precursors, in particular ortho- or para-phenylenediamines, ortho- or para-minophenols, and heterocyclic bases, which are generally known as oxidation bases. These oxidation dye precursors (oxidation bases) are colorless or weakly colored compounds which, when combined with oxidizing products, can give rise to colored compounds and dyes by a process of oxidative condensation.

It is also known that the shades obtained with these oxidation bases can be varied by combining them with couplers or coloration modifiers, the latter being chosen in particular from aromatic meta-diamines, meta-aminophenols, meta-diphenols and certain heterocyclic compounds.

The variety of molecules used as oxidation bases and couplers allows a wide range of colors to be obtained.

The so-called “permanent” coloration obtained by means of these oxidation dyes should moreover satisfy a certain number of requirements. Thus, it should allow shades to be obtained in the desired intensity and it should have good staying power with respect to external agents (light, bad weather, washing, permanent-waving, perspiration or rubbing).

The dyes should also allow gray hair to be covered and, finally, they should be as unselective as possible, i.e. they should allow only the smallest possible differences in coloration to be obtained along the same keratin fiber, which may indeed be differently sensitized (i.e. damaged) between its end and its root.

It has already been proposed, especially in patent applications JP-11-158 046, JP-11-158 047 and JP-11-158 048 to use compositions for the oxidation dyeing of keratin fibers, containing substituted para-phenylenediamine derivatives as oxidation dye precursors. However, the colorations obtained by using these compositions are not always sufficiently intense, chromatic or resistant to the various attacking factors to which the hair may be subjected.

However, the Applicant has now discovered that it is possible to obtain novel oxidation dyes, which are capable of giving chromatic, intense, attractive colorations in varied shades, which are relatively unselective and resistant to the various attacking factors to which the fibers may be subjected, by combining at least one oxidation base chosen from certain para-phenylenediamine derivatives of formula (I) defined below and the addition salts thereof with an acid, and at least one polymer chosen from those of the formulae defined below.

This discovery forms the basis of the present invention.

Thus, a first subject of the invention is a composition for the oxidation dyeing of keratin fibers, in particular of human keratin fibers such as the hair, characterized in that it comprises, in a medium that is suitable for dyeing:

(A) at least one oxidation base chosen from the substituted para-phenylenediamine derivatives of formula (I) below, and the addition salts thereof with an acid:

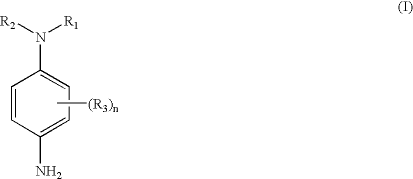

in which:

R₁ and R₂ can take one of the following meanings i) to v) below:

i) R₁ and R₂ simultaneously represent a —(CH₂)₂CHOHCH₂OH radical; or

ii) R₁ represents a —CH₂(CHOH)₄CH₂OH radical and R₂ represents a hydrogen atom, an alkyl or aryl radical or a heterocycle; or

iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;

iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;

v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;

R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;

said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;

said alkoxy radicals containing from 1 to 25 carbon atoms and possibly being linear, branched or cyclic;

said aryl radicals containing from 6 to 26 carbon atoms and possibly being substituted with one or more radicals chosen from alkyl, substituted alkyl and alkoxy radicals;

the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;

n is an integer between 0 and 4; it being understood that when n is greater than 1, then the radicals R₃ may be identical or different and may together form a 3-, 4-, 5- or 6-membered saturated or unsaturated ring; with the proviso that:

1) when R₁ and R₂ have the meanings defined in point v), then the compounds of formula (I) do not contain more than 3 hydroxyl radicals;

2) when R₁ and R₂ have the meanings defined in point v) and when R₁ and R₂ form a pyrrolidine ring substituted with a carbamoyl radical on the carbon in the position alpha to the nitrogen atom to which they are attached, then n is other than 0; or the pyrrolidine ring bears at least two substituents;

3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the β position relative to the nitrogen atom and relative to the carbon bearing said hydroxymethyl substituent; or alternatively when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;

4) when R₁ and R₂ have the meanings defined in point iii), the compounds of formula (I) must satisfy at least one of the following four conditions:

a) irrespective of the value of n, the alkylene ring formed by the radical R₂ comprises a substituent in addition to the radical R₁; or

b) n is greater than 1; or

c) when n is equal to 1, then R₃ represents an aryl radical or a heterocycle; or

d) when n is equal to 0 or 1, then R₁ represents an aryl radical, a heterocycle or a substituted alkyl radical other than a monohydroxyalkyl radical;

5) R₁ and R₂ form a ring other than piperazine or diazacycloheptane;

(B) at least one polymer chosen from:

(i) amphoteric polymers,

(ii) cationic polymers containing repeating units of structures (II) or (III) below, and

(iii) polymers other than the above, which are amphiphilic and comprise at least one fatty chain,

Formula (II):

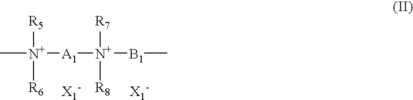

in which:

R₅, R₆, R₇ and R₈, which may be identical or different, represent aliphatic, alicyclic or arylaliphatic radicals containing from 1 to 20 carbon atoms or lower hydroxyalkylaliphatic radicals, or alternatively R₅, R₆, R₇ and R₈, together or separately, constitute, with the nitrogen atoms to which they are attached, heterocycles optionally containing a second hetero atom other than nitrogen, or alternatively R₅, R₆, R₇ and R₈ represent a linear or branched C₁-C₆ alkyl radical substituted with a nitrile, ester, acyl, amide or —CO—O—R₁₃-D or —CO—NH—R₁₃-D group in which R₁₃ is an alkylene and D is a quaternary ammonium group;

A₁ and B₁ represent polymethylene groups containing from 2 to 20 carbon atoms which may be linear or branched, saturated or unsaturated and which may contain, linked to or intercalated in the main chain, one or more aromatic rings, or one or more oxygen or sulfur atoms or sulfoxide, sulfone, disulfide, amino, alkylamino, hydroxyl, quaternary ammonium, ureido, amide or ester groups, and

X₁⁻ denotes an anion derived from a mineral or organic acid;

A₁, R₅ and R₇ may form with the two nitrogen atoms to which they are attached a piperazine ring; in addition, if A₁ denotes a linear or branched, saturated or unsaturated alkylene or hydroxyalkylene radical, B₁ can also denote a group —(CH₂)ₙ—CO-T-OC—(CH₂)ₙ— in which n is between 1 and 100 and preferably between 1 and 50, and T denotes:

a) a glycol residue of formula: —O-Z-O—, in which Z denotes a linear or branched hydrocarbon-based radical or a group corresponding to one of the following formulae:

—(CH₂—CH₂—O)x-CH₂—CH₂—

—[CH₂—CH(CH₃)—O]y-CH₂—CH(CH₃)—

in which x and y denote an integer from 1 to 4, representing a defined and unique degree of polymerization, or any number from 1 to 4 representing an average degree of polymerization;

b) a bis-secondary diamine residue such as a piperazine derivative;

c) a bis-primary diamine residue of formula: —NH—Y—NH—, in which Y denotes a linear or branched hydrocarbon-based radical, or alternatively the divalent radical

—CH₂—CH₂—S—S—CH₂—CH₂—;

d) a ureylene group of formula: —NH—CO—NH—; X₁⁻ is an anion derived from a mineral or organic acid and preferably chlorine or bromine;

Formula (III)

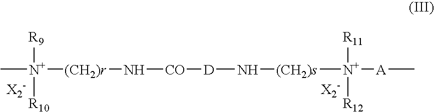

in which:

R₉, R₁₀, R₁₁ and R₁₂, which may be identical or different, represent a hydrogen atom or a methyl, ethyl, propyl, β-hydroxyethyl, β-hydroxypropyl or —CH₂CH₂(OCH₂CH₂)ₚOH radical, in which p is equal to 0 or to an integer between 1 and 6,

with the proviso that R₉, R₁₀, R₁₁ and R₁₂ do not simultaneously represent a hydrogen atom,

r and s, which may be identical or different, are integers between 1 and 6,

D is zero or denotes a group —(CH₂)_{q}—CO— in which q is zero or equal to an integer between 1 and 34,

A denotes a dihalide radical or, preferably, represents

—CH₂—CH₂—O—CH₂—CH₂—,

X₂⁻ denotes an anion derived from a mineral or organic acid, preferably a halogen atom.

The addition salts with an acid of the substituted para-phenylenediamine derivatives of formula (I) that may be used in the dye compositions according to the invention are chosen especially from the hydrochlorides, hydrobromides, sulfates, tartrates, lactates and acetates.

The dye composition in accordance with the invention thus defined gives, after mixing with an oxidizing composition, chromatic, intense, attractive colorations in varied shades, with low selectivity and excellent resistance properties not only with respect to atmospheric agents such as light and bad weather, but also with respect to perspiration and the various treatments to which the hair may be subjected.

Another subject of the invention relates to a ready-to-use composition for the oxidation dyeing of keratin fibers, which comprises, in a medium that is suitable for dyeing, at least one substituted para-phenylenediamine derivative of formula (I), at least one polymer chosen from (i) amphoteric polymers, (ii) cationic polymers containing repeating units of structure (II) or (III) defined above, and (iii) polymers other than the above comprising at least one fatty chain, and at least one oxidizing agent.

For the purposes of the present invention, the expression “ready-to-use composition” means any composition intended to be applied immediately to keratin fibers.

The invention also relates to a process for the oxidation dyeing of keratin fibers, and in particular of human keratin fibers such as the hair, which consists in applying to the fibers a dye composition containing, in a medium that is suitable for dyeing, at least one substituted para-phenylenediamine derivative of formula (I) in combination with at least one polymer chosen from amphoteric polymers, cationic polymers containing repeating units of formula (II) or (III) or amphiphilic polymers comprising at least one fatty chain, the color being developed at alkaline, neutral or acidic pH with the aid of a composition containing at least one oxidizing agent, which is mixed just at the time of use to the dye composition, or which is applied sequentially without intermediate rinsing.

A subject of the invention is also multi-compartment dyeing devices or “kits” for the oxidation dyeing of keratin fibers, in particular of human keratin fibers such as the hair.

A device comprises at least one compartment containing at least one substituted para-phenylenediamine derivative of formula (I) in combination with at least one polymer chosen from amphoteric polymers, cationic polymers containing repeating units of formula (II) or (III) or polymers other than the above containing at least one fatty chain, and a second compartment contains an oxidizing agent.

Another device comprises at least one compartment containing at least one substituted para-phenylenediamine derivative of formula (I) optionally in combination with at least one polymer chosen from amphoteric polymers, cationic polymers containing repeating units of formula (II) or (III) or amphiphilic polymers comprising at least one fatty chain, and a second compartment contains an oxidizing agent in combination with at least one polymer chosen from amphoteric polymers, cationic polymers containing repeating units of formula (II) or (III) or amphiphilic polymers comprising at least one fatty chain.

A final device comprises at least one compartment containing at least one substituted para-phenylenediamine derivative of formula (I), at least one other compartment comprising at least one polymer chosen from amphoteric polymers, cationic polymers containing repeating units of formula (II) or (III) or amphiphilic polymers comprising at least one fatty chain,

and at least one other compartment containing an oxidizing agent.

However, other characteristics, aspects, subjects and advantages of the invention will emerge even more clearly on reading the description and the examples that follow.

According to the present invention, the substituted para-phenylenediamine derivatives that are preferably used are those of formula (I) above in which:

R₁ and R₂ can take one of the meanings i) to v) below:

i) R₁ and R₂ simultaneously represent a —(CH₂)₂CHOHCH₂OH radical; or

ii) R₁ represents a —CH₂ (CHOH)₄CH₂OH radical and R₂ represents a hydrogen atom or an alkyl radical; or

iv) R₁ represents a radical —(CH₂CH₂O)ₚR₄ in which p is an integer between 2 and 8 inclusive, R₄ and R₂, which may be identical or different, represent a hydrogen atom or an alkyl radical;

v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;

R₃ represents a halogen atom, an alkyl or aryl radical or a heterocycle,

n is an integer equal to 0, 1 or 2.

More particularly, according to the invention, in formula (I), R₁ and R₂ form a pyrrolidine heterocycle.

Among the substituted para-phenylenediamine derivatives of formula (I) above that may be mentioned most particularly are 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7-amino-1-methylindole and 1-N-(hydroxyethyloxyethyl)-1-N-(2′,3′,4′,5′,6′-pentahydroxyhexyl)-3-ethyl-para-phenylenediamine, and the addition salts thereof with an acid, and among which, the ones that are even more particularly preferred are:

1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

Mention may also be made most particularly of 1-N-(3′,4′-dihydroxybutyl)-5-aminoindoline, 1-(2′-hydroxyethyl)-2-methyl-5-aminoindoline, 1-methyl-2′-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxyethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxy-ethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis-(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7 isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline and 6-amino-1-bromo-2,2-dimethyl-4-mercapto-7-isopropyloxy-1,2,3,4-tetrahydroquinoline, and the addition salts thereof with an acid.

Mention may also be made most particularly of 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2′″,4′″-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxyethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyloxyethyloxyethyloxyethyloxyethyloxyethyl)-3-methoxy-para-phenylenediamine, 1-N,N-bis(hydroxyethyloxyethyloxyethyloxyethyloxyethyloxyethyloxyethyloxyethyl)-3-methyl-para-phenylenediamine, 1,N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid,

and among which, the ones that are even more particularly preferred are:

1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrollidine, 1-(4′-amino-3′-phenoxyphenyl)-3-methylsulfonamidopyrrolidine, 1-(4′-aminophenyl)-2-(4∝-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxyethyloxyethyloxyethyl)-3-methyl-para-phenylenediamine and 1-N,N-bis(benzyloxyethyloxyethyloxyethyl)-3-isopropyl-para-phenylenediamine, and the addition salts thereof with an acid.

The para-phenylenediamine derivative(s) of formula (I) used as oxidation base in the dye composition in accordance with the invention is (are) present in concentrations ranging from 0.0001% to 20%, preferably from 0.001% to 15% and even more particularly from 0.01% to 10% by weight relative to the total weight of the composition.

Cationic Polymers

The cationic polymers containing repeating units of formula (II) preferably have a number-average molecular mass generally between 1 000 and 100 000.

Polymers of this type are especially described in French patents 2 320 330, 2 270 846, 2 316 271, 2 336 434 and 2 413 907 and patents U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

According to the invention, among these cationic polymers containing units of formula (II), those more particularly used are those consisting of repeating units corresponding to formula (IV) below:

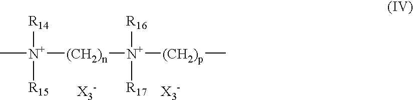

in which R₁₄, R₁₅, R₁₆ and R₁₇, which may be identical or different, denote an alkyl or hydroxyalkyl radical containing from 1 to 4 carbon atoms approximately, n and p are integers ranging from 2 to 20 approximately, and X₃⁻ is an anion derived from a mineral or organic acid.

More particularly, it is also preferred to use the polymer containing units of formula (IV) in which R₁₄, R₁₅, R₁₆ and R₁₇ denote a methyl radical, n and p are equal to 6 and 3, respectively, and X₃⁻ is equal to Cl⁻; this polymer has the INCI name: Hexadimethrine Chloride.

The cationic polymers containing repeating units of formula (III) are especially described in patent application EP-A-122 324 and may be prepared according to the processes described in U.S. Pat. Nos. 4,157,388, 4,390,689, 4,702,906 and 4,719,282.

Among these polymers, it is more particularly preferred according to the invention to use those consisting of repeating units corresponding to formula (V) below:

—[—N⁺(CH₃)₂—(CH₂)ᵣ—NH—CO-D-NH—(CH₂)ᵣ—N⁺(CH₃)₂—(CH₂)₂—O—(CH₂)₂—]—2X⁻ (V)

in which r denotes an integer ranging from 1 to 6 approximately, D may be zero or may represent a group —(CH₂)_{q}—CO— in which q denotes a number equal to 4 or to 7, and X⁻ is an anion derived from a mineral or organic acid, and preferably with a molecular mass, measured by carbon-13 NMR, of less than 100 000.

Among the cationic polymers of formula (V) which are even more particularly preferred are those for which:

a) D represents a group —(CH₂)₄—CO—, X denotes a chlorine atom, the molecular mass, measured by carbon-13 NMR (C¹³ NMR), being about 5 600; a polymer of this type is proposed by the company Miranol under the name Mirapol-AD1,

b) D represents a group —(CH₂)₇—CO— and X denotes a chlorine atom, the molecular mass, measured by carbon-13 NMR (C¹³NMR) being about 8100; a polymer of this type is proposed by the company Miranol under the name Mirapol-AZ1,

c) D denotes the value zero and X denotes a chlorine atom, the molecular mass, measured by carbon-13 NMR (C¹³ NMR), being about 25 500; a polymer of this type is sold by the company Miranol under the name Mirapol-A15,

d) a “block copolymer” formed from units corresponding to the polymers described in paragraphs a) and c), sold by the company Miranol under the names Mirapol-9 (C¹³ NMR molecular mass of about 7 800) Mirapol-175 (C¹³ NMR molecular mass of about 8 000) and Mirapol-95 (C¹³ NMR molecular mass of about 12 500).

Even more particularly, the polymer which is preferred according to the invention is the polymer of formula (V) in which D denotes the value zero, X denotes a chlorine atom, the molecular mass, measured by carbon-13 NMR (C¹³ NMR), being about 25 500.

According to the invention, the cationic polymer(s) can represent from 0.01% to 10% by weight approximately relative to the total weight of the composition, preferably from 0.05% to 5% and even more preferentially from 0.1% to 3%.

Amphoteric polymers

The amphoteric polymers which can be used in accordance with the present invention can be chosen from polymers containing units K and M distributed randomly in the polymer chain, in which K denotes a unit derived from a monomer containing at least one basic nitrogen atom and M denotes a unit derived from an acid monomer containing one or more carboxylic or sulfonic groups, or alternatively K and M can denote groups derived from carboxybetaine or sulfobetaine zwitterionic monomers;

K and M can also denote a cationic polymer chain containing primary, secondary, tertiary or quaternary amine groups, in which at least one of the amine groups bears a carboxylic or sulfonic group connected via a hydrocarbon-based radical, or alternatively K and M form part of a chain of a polymer containing an α,β-dicarboxylic ethylene unit in which one of the carboxylic groups has been made to react with a polyamine containing one or more primary or secondary amine groups.

The amphoteric polymers which are more particularly preferred are chosen from the following polymers:

(1) Polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylates and acrylates, dialkylaminoalkylmethacrylamides and acrylamides. Such compounds are described in U.S. Pat. No. 3,836,537. Mention may also be made of the sodium acrylate/acrylamidopropyltrimethylammonium chloride copolymer sold under the name Polyquart KE 3033 by the company Henkel.

The substituted vinyl compound which contains at least one basic atom can also be a dialkyldiallylammonium salt such as dimethyldiallylammonium chloride. The copolymers of acrylic acid and of the latter monomer are sold under the names Merquat 280, Merquat 295 and Merquat Plus 3330 by the company Calgon.

(2) Polymers containing units derived from:

a) at least one monomer chosen from acrylamides and methacrylamides substituted on the nitrogen with an alkyl radical,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid, fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer such as the products sold under the name Amphomer or Lovocryl 47 by the company National Starch are particularly used.

(3) Crosslinked and alkylated polyamino amides partially or totally derived from polyamino amides of general formula:

—[—CO—R₁₈—CO-Z—] (VI)

in which R₁₈ represents a divalent radical derived from a saturated dicarboxylic acid, a mono- or dicarboxylic aliphatic acid containing an ethylenic double bond, an ester of a lower alkanol, having 1 to 6 carbon atoms of these acids or a radical derived from the addition of any one of said acids to a bis(primary) or bis(secondary) amine, and Z denotes a bis(primary), mono- or bis(secondary) polyalkylene-polyamine radical and preferably represents:

a) in proportions of from 60 to 100 mol %, the radical

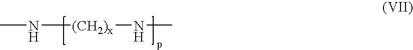

where x=2 and p=2 or 3, or alternatively x=3 and p=2 this radical being derived from diethylenetriamine, from triethylenetetraamine or from dipropylenetriamine;

b) in proportions of from 0 to 40 mol %, the radical (VII) above, in which x=2 and p=1 and which is derived from ethylenediamine, or the radical derived from piperazine:

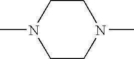

c) in proportions of from 0 to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.

(4) Polymers containing zwitterionic units of formula:

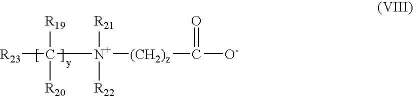

in which R₂₃ denotes a polymerizable unsaturated group such as an acrylate, methacrylate, acrylamide or methacrylamide group, y and z represent an integer from 1 to 3, R₁₉ and R₂₀ represent a hydrogen atom, methyl, ethyl or propyl, R₂₁ and R₂₂ represent a hydrogen atom or an alkyl radical such that the sum of the carbon atoms in R₂₁ and R₂₂ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of butyl methacrylate/dimethyl carboxymethylammonio ethyl methacrylate such as the product sold under the name Diaformer Z301 by the company Sandoz.

(5) Polymers derived from chitosan containing monomer units corresponding to the following formulae (IX), (X) and (XI):

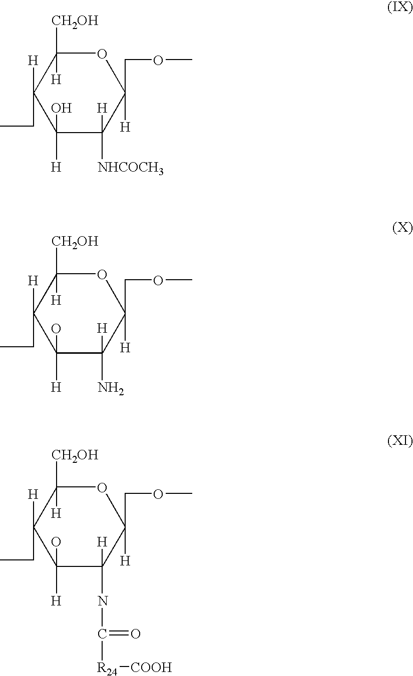

the unit (IX) being present in proportions of between 0 and 30%, the unit (X) in proportions of between 5 and 50% and the unit (XI) in proportions of between 30 and 90%, it being understood that, in this unit (XI), R₂₄ represents a radical of formula:

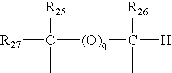

in which if q=0, R₂₅, R₂₆ and R₂₇, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the radicals R₂₅, R₂₆ and R₂₇ being, in this case, a hydrogen atom; or, if q=1, R₂₅, R₂₆ and R₂₇ each represent a hydrogen atom, as well as the salts formed by these compounds with bases or acids.

(6) Polymers derived from the N-carboxyalkylation of chitosan, such as N-carboxymethylchitosan or N-carboxybutylchitosan sold under the name “Evalsan” by the company Jan Dekker.

(7) Polymers corresponding to the general formula (XII) such as those described, for example, in French patent

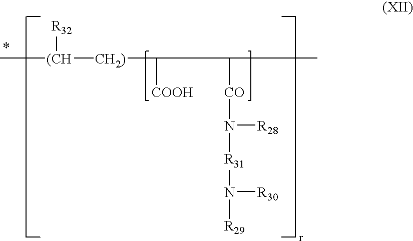

in which R₃₂ represents a hydrogen atom, a CH₃O, CH₃CH₂O or phenyl radical, R₂₈ denotes hydrogen or a lower alkyl radical such as methyl or ethyl, R₂₉ denotes hydrogen or a lower alkyl radical such as methyl or ethyl, R₃₀ denotes a lower alkyl radical such as methyl or ethyl or a radical corresponding to the formula: —R₃₁—N(R₂₉)₂, R₃₁ representing a —CH₂—CH₂—, —CH₂—CH₂—CH₂— or —CH₂—CH(CH₃)— group, R₂₉ having the meanings mentioned above, as well as the higher homologs of these radicals and containing up to 6 carbon atoms, r is such that the molecular weight is between 500 and 6 000 000 and preferably between 1 000 and 1 000 000.

(8) Amphoteric polymers of the type -D-X-D-X chosen from:

a) polymers obtained by the action of chloroacetic acid or sodium chloroacetate on compounds containing at least one unit of formula:

-D-X-D-X-D- (XIII)

where D denotes a radical

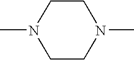

and X denotes the symbol E or E′, E or E′, which may be identical or different, denote a divalent radical which is an alkylene radical containing a straight or branched chain containing up to 7 carbon atoms in the main chain, which is unsubstituted or substituted with hydroxyl groups and which can contain, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;

b) polymers of formula:

-D-X-D-X- (XIV)

in which D denotes a radical

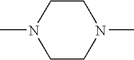

and X denotes the symbol E or E′ and at least once E′; E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The amphoteric polymers which are particularly preferred according to the invention are those of family (1).

According to the invention, the amphoteric polymer(s) can represent from 0.01% to 10% by weight, preferably from 0.05% to 5% by weight and even more preferably from 0.1% to 3% by weight, relative to the total weight of the composition.

Fatty-Chain Amphiphilic Polymers

The polymers other than the above are amphiphilic and contain at least one fatty chain; they are also known as associative polymers; they may be of nonionic, anionic or cationic type.

Among the amphiphilic polymers comprising at least one fatty chain and of anionic type, mention may be made of:

(I) those comprising at least one hydrophilic unit and at least one fatty-chain allyl ether unit, more particularly those of which the hydrophilic unit consists of an ethylenic unsaturated anionic monomer, even more particularly a vinylcarboxylic acid and most particularly an acrylic acid, a methacrylic acid or mixtures thereof, the allyl ether unit containing a fatty chain corresponding to the monomer of formula (XV) below:

CH₂═CR′CH₂OBₙR (XV)

in which R′ denotes H or CH₃, B denotes an ethyleneoxy radical, n is zero or denotes an integer ranging from 1 to 100, R denotes a hydrocarbon-based radical chosen from alkyl, arylalkyl, aryl, alkylaryl and cycloalkyl radicals, containing 8 to 30 carbon atoms, preferably 10 to 24 carbon atoms and even more particularly from 12 to 18 carbon atoms. A unit of formula (XV) which is more particularly preferred is a unit in which R′ denotes H, n is equal to 10 and R denotes a stearyl (C₁₈) radical.

Anionic amphiphilic polymers of this type are described and prepared, according to an emulsion polymerization process, in patent EP-0 216 479.

Among these fatty-chain anionic polymers, those which are particularly preferred according to the invention are polymers formed from 20% to 60% by weight of acrylic acid and/or of methacrylic acid, from 5% to 60% by weight of lower alkyl (meth)acrylates, from 2% to 50% by weight of allyl ether containing a fatty chain of formula (XV), and from 0% to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl ether (Steareth-10), in particular those sold by the company Allied Colloids under the names Salcare SC 80 and Salcare SC 90, which are aqueous 30% emulsions of a crosslinked terpolymer of methacrylic acid, of ethyl acrylate and of steareth-10 allyl ether (40/50/10).

(II) those comprising at least one hydrophilic unit of unsaturated olefinic carboxylic acid type, and at least one hydrophobic unit of (C₁₀-C₃₋₀)alkyl ester of unsaturated carboxylic acid type.

Preferably, these polymers are chosen from those in which the hydrophilic unit of unsaturated olefinic carboxylic acid type corresponds to the monomer of formula (XVI) below:

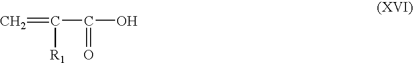

in which R₁ denotes H or CH₃ or C₂H₅, that is to say acrylic acid, methacrylic acid or ethacrylic acid units, and in which the hydrophobic unit of (C₁₀-C₃₀) alkyl ester of unsaturated carboxylic acid type corresponds to the monomer of formula (XVII) below:

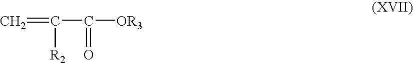

in which R₂ denotes H or CH₃ or C₂H₅ (that is to say acrylate, methacrylate or ethacrylate units) and preferably H (acrylate units) or CH₃ (methacrylate units), R₃ denoting a C₁₀-C₃₀ and preferably Cl₂—C₂₂ alkyl radical.

(C₁₀-C₃₀) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the fatty-chain anionic polymers of this type which will be used more particularly are polymers formed from a monomer mixture comprising:

(i) essentially acrylic acid,

(ii) an ester of formula (XVI) described above in which R₂ denotes H or CH₃, R₃ denoting an alkyl radical containing from 12 to 22 carbon atoms,

(iii) and a crosslinking agent, which is a well-known copolymerizable polyethylenic unsaturated monomer, for instance diallyl phthalate, allyl (meth)acrylate, divinylbenzene, (poly)ethylene glycol dimethacrylate and methylenebisacrylamide.

Among fatty-chain anionic polymers of this type which will be used more particularly are those consisting of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among said above polymers, those most particularly preferred according to the present invention are the products sold by the company Goodrich under the trade names Pemulen TR1, Pemulen TR2 and Carbopol 1382, and even more preferentially Pemulen TR1, and the product sold by the company S.E.P.P.I.C under the name Coatex SX.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608 by the company Newphase Technologies.

(IV) acrylic terpolymers comprising:

(a) about 20% to 70% by weight of a carboxylic acid containing α,β-monoethylenic unsaturation,

(b) about 20% to 80% by weight of a non-surfactant monomer containing α, β-monoethylenic unsaturation other than (a),

(c) about 0.5% to 60% by weight of a nonionic monourethane which is the product of reaction of a monohydric surfactant with a monoisocyanate containing monoethylenic unsaturation,

such as those described in patent application EP-A-0 173 109 and more particularly the terpolymer described in example 3, namely a methacrylic acid/methyl acrylate/behenyl dimethyl-metaisopropenylbenzylisocyanate ethoxylated (40 EO) terpolymer, as an aqueous 25% dispersion.

(V) copolymers comprising among their monomers a carboxylic acid containing α,β-monoethylenic unsaturation and an ester of a carboxylic acid containing α,β-monoethylenic unsaturation and of a (C₈-C₃₀)oxyalkylenated fatty alcohol.

Preferentially, these compounds also comprise as monomer an ester of a carboxylic acid containing α,β-monoethylenic unsaturation and of a C₁-C₄ alcohol.

An example of a compound of this type which may be mentioned is Aculyn 22 sold by the company Rohm & Haas, which is a methacrylic acid/ethyl acrylate/stearyl methacrylate oxyalkylenated terpolymer.

The fatty-chain amphiphilic polymers of nonionic type which are used according to the invention are preferably chosen from:

(1) celluloses modified with groups comprising at least one fatty chain; examples which may be mentioned include:

hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS(C₁₆ alkyls) sold by the company Aqualon, or the product Bermocoll EHM 100 sold by the company Berol Nobel,

those modified with alkylphenyl polyalkylene glycol ether groups, such as the product Amercell Polymer HM-1500 (nonylphenyl polyethylene glycol (15) ether) sold by the company Amerchol.

(2) hydroxypropylguars modified with groups comprising at least one fatty chain, such as the product Esaflor HM 22 (C₂₂ alkyl chain) sold by the company Lamberti, and the products RE210-18 (C₁₄ alkyl chain) and RE205-1 (C₂₀ alkyl chain) sold by the company Rhône-Poulenc.

(3) copolymers of vinylpyrrolidone and of fatty-chain hydrophobic monomers;

examples which may be mentioned include:

the products Antaron V216 or Ganex V216 (vinylpyrrolidone/hexadecene copolymer) sold by the company I.S.P

the products Antaron V220 or Ganex V220 (vinylpyrrolidone/eicosene copolymer) sold by the company I.S.P.

(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208.

(5) copolymers of hydrophilic methacrylates or acrylates and of hydrophobic monomers comprising at least one fatty chain, such as, for example, the polyethylene glycol methacrylate/lauryl methacrylate copolymer.

(6) polymers with an aminoplast ether skeleton containing at least one fatty chain, such as the Pure Thix compounds sold by the company Sud-Chemie.

(7) polyurethane polyethers comprising in their chain both hydrophilic blocks usually of polyoxyethylenated nature and hydrophobic blocks which may be aliphatic sequences alone and/or cycloaliphatic and/or aromatic sequences.

Preferably, the polyether polyurethanes comprise at least two hydrocarbon-based lipophilic chains containing from 6 to 30 carbon atoms, separated by a hydrophilic block, the hydrocarbon-based chains possibly being pendent chains, or chains at the end of the hydrophilic block. In particular, it is possible for one or more pendent chains to be included. In addition, the polymer may comprise a hydrocarbon-based chain at one end or at both ends of a hydrophilic block.

The polyether polyurethanes may be multiblock, in particular in triblock form. Hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The nonionic fatty-chain polyether polyurethanes may be triblock copolymers in which the hydrophilic block is a polyoxyethylenated chain comprising from 50 to 1 000 oxyethylene groups. The nonionic polyether polyurethanes comprise a urethane linkage between the hydrophilic blocks, whence arises the name.

By extension, also included among the nonionic fatty-chain polyether polyurethanes are those in which the hydrophilic blocks are linked to the lipophilic blocks via other chemical bonds.

As examples of nonionic fatty-chain polyether polyurethanes which may be used in the invention, use may also be made of Rheolate 205 containing a urea function, sold by the company Rheox, or the Rheolates 208, 204 or 212, and also Acrysol RM 184, Aculyn or Acrysol 44 and Aculyn or Acrysol 46 from the company Rohm & Haas [Aculyn 46 is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44 is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Mention may also be made of the product Elfacos T210 containing a C₁₂₋₁₄ alkyl chain, and the product Elfacos T212 containing a C₁₆ alkyl chain, from Akzo.

The product DW 1206B from Rohm & Haas containing a C₂₀ alkyl chain and urethane linkage, sold at a solids content of 20% in water, may also be used.

It is also possible to use solutions or dispersions of these polymers, especially in water or in aqueous-alcoholic medium. Examples of such polymers which may be mentioned are Rheolate 255, Rheolate 278 and Rheolate 244 sold by the company Rheox. The products DW 1206F and DW 1206J sold by the company Rohm & Haas may also be used.

The polyether polyurethanes which may be used according to the invention are in particular those described in the article by G. Formum, J. Bakke and Fk. Hansen—Colloid Polym. Sci 271, 380.389 (1993).

The amphiphilic polymers of cationic type comprising at least one fatty chain which are used in the present invention are preferably chosen from quaternized cellulose derivatives and polyacrylates containing noncyclic amino side groups.

The quaternized cellulose derivatives are, in particular,

quaternized celluloses modified with groups comprising at least one fatty chain, such as alkyl, aralalkyl or alkylaryl groups containing at least 8 carbon atoms, or mixtures thereof,

quaternized hydroxyethylcelluloses modified with groups comprising at least one fatty chain, such as alkyl, aralalkyl or alkylaryl groups containing at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl or anthryl groups.

Examples of quaternized alkylhydroxyethylcelluloses containing C₈-C₃₀ fatty chains which may be mentioned include the products Quatrisoft LM 200, Quatrisoft LM-X 529-18-A, Quatrisoft LM-X 529-18B (C₁₂ alkyl) and Quatrisoft LM-X 529 8 (C₁₈ alkyl) sold by the company Amerchol, and the products Crodacel QM, Crodacel QL (C₁₂ alkyl) and Crodacel QS (C₁₈ alkyl) sold by the company Croda.

The polyacrylates containing quaternized or nonquaternized amino side groups contain, for example, hydrophobic groups of the type such as Steareth 20 (polyoxyethylenated(20) stearyl alcohol).

Examples of polyacrylates containing amino side chains which may be mentioned include the polymers 8781-121B and 9492-103 sold by the company National Starch.

In the oxidation dye composition according to the invention, among the fatty-chain amphiphilic polymers, it is preferred to use an amphiphilic polymer of nonionic or cationic type comprising at least one fatty chain.

According to the invention, the amphiphilic polymer(s) comprising at least one fatty chain may represent about 0.01% to 10% by weight relative to the total weight of the composition. Preferably, this amount ranges from about 0.1% to 5% by weight.

Couplers

Preferably, the compositions of the invention contain at least one coupler.

Among these couplers, mention may be made especially of meta-phenylenediamines, metaminophenols, meta-diphenols and heterocyclic couplers such as, for example, indole derivatives, indoline derivatives, benzimidazole derivatives, benzomorpholine derivatives, sesamol derivatives, pyridine, pyrimdine and pyrazole derivatives, and the addition salts thereof with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

The coupler(s) may be present in said composition according to the invention in a concentration of between 0.0001% and 15% by weight relative to the total weight of the composition.

The dye composition in accordance with the invention may also contain at least one additional oxidation base other than the substituted para-phenylenediamine derivatives of formula (I) and/or at least one direct dye.

Among the additional oxidation bases that may be used according to the invention, mention may be made of para-phenylenediamine, para-tolylenediamine, 2-hydroxyethyl-para-phenylenediamine, 1-N,N-bis(2-hydroxyethyl)-para-phenylenediamine, para-aminophenols such as 3-methyl-4-aminophenol and 4-aminophenol, ortho-phenylenediamines, ortho-aminophenols, double bases, heterocyclic bases, for instance pyrimidines such as 2,4,5,6-tetraminopyrimidine, or pyrazoles, such as 1-(2-hydroxyethyl)-4,5-diaminopyrazole.

The additional oxidation base(s) may be present in a concentration of between 0.0001% and 15% by weight relative to the total weight of said composition.

Medium

The medium for the composition that is suitable for dyeing is preferably an aqueous medium consisting of water and can advantageously contain cosmetically acceptable organic solvents including, more particularly, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers such as, for example, diethylene glycol monoethyl ether or monobutyl ether. The solvents may then be present in concentrations of between about 1% and about 40% and preferably between about 5% and 30% by weight relative to the total weight of the composition.

The composition according to the invention may also contain an effective amount of other agents that are previously known elsewhere in oxidation dyeing, such as various common adjuvants, for instance sequestering agents such as EDTA and etidronic acid, UV screening agents, waxes, volatile or nonvolatile, cyclic or linear or branched silicones, that are organomodified (especially with amine groups) or unmodified, preserving agents, ceramides, pseudoceramides, plant, mineral or synthetic oils, vitamins or provitamins, for instance panthenol, opacifiers, thickeners such as crosslinked polyacrylic acids or hydroxyalkylcelluloses, etc.

Said composition may also contain reducing agents or antioxidants. These may be chosen in particular from sodium sulfite, thioglycolic acid, thiolactic acid, sodium bisulfite, dehydroascorbic acid, hydroquinone, 2-methylhydroquinone, tertbutylhydroquinone and homogentisic acid, and they are then generally present in amounts ranging from about 0.05 to 1.5% by weight, relative to the total weight of the composition.

The composition according to the invention can also contain one or more fatty alcohols, these fatty alcohols being introduced in pure form or as a mixture. Among these, mention may be made more particularly of lauryl alcohol, cetyl alcohol, stearyl alcohol and oleyl alcohol and mixtures thereof. These fatty alcohols can represent from 0.001% to 20% by weight approximately relative to the total weight of the composition.

Preferably, the composition of the invention contains at least one nonionic, anionic, cationic or amphoteric surfactant in a proportion ranging from about 0.1 to about 20% by weight.

Even more preferentially, said composition contains at least one nonionic surfactant.

The composition according to the invention can also contain a cationic polymer other than those of formula (II) or (III) according to the present invention, in a proportion ranging from about 0.01% to about 10% by weight.

Needless to say, a person skilled in the art will take care to select the optional additional compound(s) mentioned above, such that the advantageous properties intrinsically associated with the oxidation dye composition according to the invention are not, or are not substantially, adversely affected by the envisaged addition(s).

Oxidizing Agent

The oxidizing agent is preferably chosen from hydrogen peroxide, urea peroxide, alkali metal bromates or ferricyanides, and persalts such as perborates and persulfates. The use of hydrogen peroxide is particularly preferred. This oxidizing agent advantageously consists of an aqueous hydrogen peroxide solution whose titer can range more particularly from about 1 to about 40 volumes and even more preferentially from about 5 to about 40 volumes.

One or more redox enzymes such as 4-electron oxidoreductases (such as laccases), peroxidases and 2-electron oxidoreductases (such as uricase), where appropriate in the presence of their respective donor or cofactor, may also be used as oxidizing agent.

The pH of the dye composition or of the composition ready to be used and applied to the keratin fibers [composition resulting from mixing the dye composition according to the invention and the oxidizing composition] is generally between the values 4 and 12. It is preferably between 6 and 11, and may be adjusted to the desired value by means of acidifying or basifying agents that are well known in the prior art in the dyeing of keratin fibers.

Among the basifying agents that may be mentioned, for example, are aqueous ammonia, alkali metal carbonates, alkanolamines such as monoethanolamine, diethanolamine and triethanolamine and also derivatives thereof, oxyethylenated and/or oxypropylenated hydroxyalkylamines and ethylenediamines, sodium hydroxide, potassium hydroxide and the compounds of formula (XVIII) below:

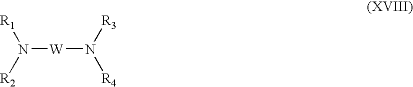

in which W is a propylene residue optionally substituted with a hydroxyl group or a C₁-C₄ alkyl radical; R₁, R₂, R₃ and R₄, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulfonic acids.

The dye composition in accordance with the invention may be in various forms, such as in the form of liquids, powders, creams or gels, that may be pressurized, or in any other form that is suitable for dyeing keratin fibers, and especially human hair.

The dyeing process according to the invention preferably consists in applying the ready-to-use composition, prepared extemporaneously at the time of use from the dye composition according to the invention and the oxidizing composition described above, to the wet or dry keratin fibers, and in leaving the composition to act for an exposure time preferably ranging from 1 to 60 minutes approximately and more preferably from 10 to 45 minutes approximately, rinsing the fibers and then optionally washing them with shampoo, followed by rinsing them again and drying them.

The examples that follow are intended to illustrate the invention.

### EXAMPLES

The dye compositions below, in accordance with the invention, were prepared:

| EXAMPLES | 1 | 2 | 3 | 4 |
|---|---|---|---|---|
| 1-(4′-Amino-3′-methyl- | 0.837 g | 0.837 g | 0.837 g | 0.837 g |
| phenyl)-4-hydroxy-2- | | | | |
| methylpyrrolidine | | | | |
| dihydrochloride | | | | |
| [substituted para- | | | | |
| phenylenediamine | | | | |
| derivative of formula | | | | |
| (I) in accordance with | | | | |
| the invention] | | | | |
| 2,4-Diamino-1-(β- | 0.723 g | 0.723 g | 0.723 g | 0.723 g |
| hydroxyethyloxy)- | | | | |
| benzene | | | | |
| dihydrochloride | | | | |
| Mirapol A15 (Miranol) | 1 g AM* | | | |
| Merquat 280 (Calgon) | | 1 g AM* | | |
| Acrylsol 44 (Rohm & | | | 1 g AM* | |
| Haas) | | | | |
| Hexadimethrine | | | | 1 g AM* |
| Chloride (Chimex) | | | | |
| Dye support (*) | qs | qs | qs | qs |
| Demineralized water qs | 100 g | 100 g | 100 g | 100 g |

*denotes Active Material
(*) Dye support
C8-C10 alkyl polyglucoside as an aqueous 60% solution, sold under the name Oramix CG 110 ® by the company
SEPPIC 3.24 g AM*
Ethanol 18.0 g
Benzyl alcohol 1.8 g
Polyethylene glycol 400 2.7 g
Pentasodium salt of diethylene-triaminepentaacetic acid as an aqueous 40% solution, sold under the name Dissoluine D-40 ® by the company Akzo 0.43 g AM*
Sodium metabisulfite 0.205 g
Aqueous ammonia containing 20.5% NH₃ 10.0 g
AM* denotes Active Material

At the time of use, each of the 4 dye compositions described above was mixed weight for weight with a 20-volumes hydrogen peroxide solution (6% by weight).

The mixtures thus prepared were applied for 30 minutes to locks of permanent-waved natural gray hair containing 90% white hairs. The locks were then rinsed, washed with a standard shampoo, rinsed again and then dried.

In the four cases, the hairs were dyed a bright blue shade.

1. A composition for the oxidation dyeing of keratin fibers, in particular of human keratin fibers such as the hair, characterized in that it comprises, in a medium that is suitable for dyeing:
(A) at least one oxidation base chosen from the substituted para-phenylenediamine derivatives of formula (I) below, and the addition salts thereof with an acid: 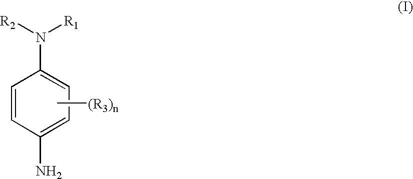
in which:
R₁ and R₂ can take one of the following meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom, an alkyl or aryl radical or a heterocycle; or
iii) R₁ represents an alkyl or aryl radical or a heterocycle and R₂ represents an alkylene radical (CH₂)ₘ— in which m is an integer equal to 2 or 3, said alkylene radical forming a ring together with the nitrogen atom, the carbon atom of the benzene ring bearing the nitrogen atom and one of the two carbon atoms of the benzene ring that are adjacent to it, it being understood that when R₁ is an alkyl or aryl radical, then either R₁ or said alkylene radical is substituted with a radical containing at least one nitrogen, oxygen or sulfur atom;
iv) R₁ represents a radical —(CH₂CH₂O)ₚR₄ in which p is an integer between 2 and 8 inclusive, R₄ and R₂, which may be identical or different, represent a hydrogen atom, an alkyl or aryl radical or a heterocycle;
v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;
R₃ represents a halogen atom, an alkyl or aryl radical, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical, a heterocycle or a heterocycle linked to the benzene ring of formula (I) via an ether or thio bond;
said alkyl radicals containing from 1 to 25 carbon atoms and possibly being linear, branched or cyclic and possibly being substituted with one or more radicals, and in this case possibly representing a mono- or polyhydroxyalkyl radical, an alkoxyalkyl radical, an aminoalkyl radical optionally substituted on the nitrogen atom, or a carboxyalkyl, alkylcarboxyalkyl, thioalkyl, alkylthioalkyl, cyanoalkyl, trifluoroalkyl, sulfoalkyl, phosphoalkyl or haloalkyl radical;
said alkoxy radicals containing from 1 to 25 carbon atoms and possibly being linear, branched or cyclic;
said aryl radicals containing from 6 to 26 carbon atoms and possibly being substituted with one or more radicals chosen from alkyl, substituted alkyl and alkoxy radicals;
the heterocycles being monocyclic or polycyclic, each ring being 3-, 4-, 5- or 6-membered and possibly containing one or more hetero atoms, it being understood that in the case of polycyclic heterocycles, at least one of the rings contains at least one hetero atom such as N, O or S;
n is an integer between 0 and 4; it being understood that when n is greater than 1, then the radicals R₃ may be identical or different and may together form a 3-, 4-, 5- or 6-membered saturated or unsaturated ring; with the proviso that:
1) when R₁ and R₂ have the meanings defined in point v), then the compounds of formula (I) do not contain more than 3 hydroxyl radicals;
2) when R₁ and R₂ have the meanings defined in point v) and when R₁ and R₂ form a pyrrolidine ring substituted with a carbamoyl radical on the carbon in the alpha position to the nitrogen atom to which they are attached, then n is other than 0; or the pyrrolidine ring bears at least two substituents;
3) when R₁ and R₂ have the meanings defined in point v) and when R₁ and R₂ form a pyrrolidine ring substituted with a hydroxymethyl radical on the carbon in the alpha position relative to the nitrogen atom to which they are attached and when n=0 or 1, then either said ring bears at least two additional substituents, or said ring bears only one second substituent other than a hydroxyl radical on the carbon located in the β position relative to the nitrogen atom and relative to the carbon bearing said hydroxymethyl substituent; or alternatively when R₁ and R₂ have the meanings defined in point v) and when R₁ and R₂ form a pyrrolidine ring substituted with a hydroxymethyl radical on the carbon located in the alpha position relative to the nitrogen atom to which they are attached, and when n=1, then R₃ is other than an alkyl or mono- or polyhydroxyalkyl radical;
4) when R₁ and R₂ have the meanings defined in point iii), the compounds of formula (I) must satisfy at least one of the following four conditions:
a) irrespective of the value of n, the alkylene ring formed by the radical R₂ comprises a substituent in addition to the radical R₁; or
b) n is greater than 1; or
c) when n is equal to 1, then R₃ represents an aryl radical or a heterocycle; or
d) when n is equal to 0 or 1, then R₁ represents an aryl radical, a heterocycle or a substituted alkyl radical other than a monohydroxyalkyl radical;
5) R₁ and R₂ form a ring other than piperazine or diazacycloheptane;
(B) at least one polymer chosen from:
(i) amphoteric polymers,
(ii) cationic polymers containing repeating units of structures (II) or (III) below, and
(iii) amphiphilic polymers comprising at least one fatty chain,
Formula (II): 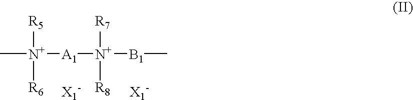
in which:
R₅, R₆, R₇ and R₅, which may be identical or different, represent aliphatic, alicyclic or arylaliphatic radicals containing from 1 to 20 carbon atoms or lower hydroxyalkyl radicals, or alternatively R₅, R₆, R₇ and R₈, together or separately, constitute, with the nitrogen atoms to which they are attached, heterocycles optionally containing a second hetero atom other than nitrogen, or alternatively R₅, R₆, R₇ and R₈ represent a linear or branched C₁-C₆ alkyl radical substituted with a nitrile, ester, acyl, amide or —CO—O—R₁₃-D or —CO—NH—R₁₃-D group in which R₁₃ is an alkylene and D is a quaternary ammonium group;
A₁ and B₁ represent polymethylene groups containing from 2 to 20 carbon atoms which may be linear or branched, saturated or unsaturated and which may contain, linked to or intercalated in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X₁⁻ denotes an anion derived from a mineral or organic acid;
A₁, R₅ and R₇ may form with the two nitrogen atoms to which they are attached a piperazine ring; in addition, if A₁ denotes a linear or branched, saturated or unsaturated alkylene or hydroxyalkylene radical, B₁ can also denote a group —(CH₂)ₙ—CO-T-OC—(CH₂)ₙ— in which n is between 1 and 100 and preferably between 1 and 50, and T denotes:
a) a glycol residue of formula: —O-Z-O—, in which Z denotes a linear or branched hydrocarbon-based radical or a group corresponding to one of the following formulae:
—(CH₂—CH₂—O)x-CH₂—CH₂
—[CH₂—CH(CH₃)—O]y-CH₂—CH(CH₃)—
in which x and y denote an integer from 1 to 4, representing a defined and unique degree of polymerization, or any number from 1 to 4 representing an average degree of polymerization;
b) a bis-secondary diamine residue such as a piperazine derivative;
c) a bis-primary diamine residue of formula: —NH—Y—NH—, in which Y denotes a linear or branched hydrocarbon-based radical, or alternatively the divalent radical
—CH₂—CH₂—S—S—CH₂—CH₂—;
d) a ureylene group of formula: —NH—CO—NH—; X₁⁻ is an anion derived from a mineral or organic acid;
Formula (III) 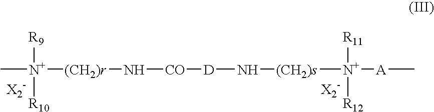
in which:
R₉, R₁₀, R₁₁ and R₁₂, which may be identical or different, represent a hydrogen atom or a methyl, ethyl, propyl, β-hydroxyethyl, β-hydroxypropyl or —CH₂CH₂(OCH₂CH₂)ₚOH radical, in which p is equal to 0 or to an integer between 1 and 6,
with the proviso that R₉, R₁₀, R₁₁ and R₁₂ do not simultaneously represent a hydrogen atom,
r and s, which may be identical or different, are integers between 1 and 6,
D is zero or denotes a group —(CH₂)_{q}—CO— in which q is zero or equal to an integer between 1 and 34,
A denotes a dihalide radical or, preferably, represents —CH₂—CH₂—O—CH₂—CH₂—,
X₂⁻ denotes an anion derived from a mineral or organic acid.

2. The composition as claimed in claim 1, characterized in that, in formula (I), R₁ and R₂ take one of the meanings i) to v) below:
i) R₁ and R₂ simultaneously represent a —(CH₂)₂CHOHCH₂OH radical; or
ii) R₁ represents a —CH₂(CHOH)₄CH₂OH radical and R₂ represents a hydrogen atom or an alkyl radical; or
iv) R₁ represents a radical —(CH₂CH₂O)ₚR₄ in which p is an integer between 2 and 8 inclusive, R₄ and R₂, which may be identical or different, represent a hydrogen atom or an alkyl radical;
v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, which is not in a meta position relative to the nitrogen atom of the heterocycle;
R₃ represents a halogen atom, an alkyl or aryl radical or a heterocycle,
n is an integer equal to 0, 1 or 2.

3. The composition as claimed in claim 1 or 2, characterized in that, in formula (I), R₁ and R₂ form a pyrrolidine heterocycle.

4. The composition as claimed in claim 1 or 2, characterized in that the substituted para-phenylenediamine derivative(s) of formula (I) is (are) chosen from: 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis-(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-β-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)-amino-7-amino-1-methylindole, 1-N-(hydroxyethyloxyethyl)-1-N-(2′,3′,4′,5′,6′-pentahydroxyhexyl)-3-ethyl-para-phenylenediamine, 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxyethyloxyethyloxyethyl-6-isopropyl-5-aminoindoline, 2-hydroxyethyl-3-methyl-5-aminoindoline, 2-hydroxyethyloxyethyloxyethyl)-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, the dimethyl ester 6-amino-1-methyl-1,2.,3,4-tetrahydrofuro-[2,3,h]-quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxyethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline, 6-amino-1-bromo-2,2-dimethyl-4-mercapto-7-isopropyloxy-1,2,3,4-tetrahydroquinoline, 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic acid), 1-(4′-amino-3′-acetylaminophenyl)-3-hydroxymethylpyrrolidine, 7-amino-4-(2′-methyl)-pyrrolidinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2″,4″-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxyethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3-4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(methoxyethyloxyethyloxyethyloxyethyloxyethyloxyethyloxyethyl)-3-methoxy-para-phenylenediamine, 1-N,N-bis(hydroxyethyloxyethyloxyethyloxyethyloxyethyloxyethyloxyethyloxyethyl)-3-methyl-para-phenylenediamine, 1-N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine and 1-N,N-bis(benzyloxyethyloxyethyloxyethyl)-3-isopropyl-para-phenylenediamine, and the addition salts thereof with an acid.

5. The composition as claimed in claim 4, characterized in that they are chosen from: 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-penta-hydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine and 1-N-(hydroxyethyloxyethyl)-1-N-(2′,3′,4′,5′,6′-pentahydroxyhexyl)-3-ethyl-para-phenylenediamine, and the addition salts thereof with an acid.

6. The composition as claimed in claim 4, characterized in that they are chosen from: 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxyethyloxyethyloxyethyl)-3-methyl-para-phenylenediamine and 1-N,N-bis(benzyloxyethyloxyethyloxyethyl)-3-isopropyl-para-phenylenediamine.

7. The composition as claimed in any one of the preceding claims, characterized in that the addition salts with an acid are chosen from the hydrochlorides, hydrobromides, sulfates, tartrates, lactates and acetates.

8. The composition as claimed in any one of the preceding claims, characterized in that the substituted para-phenylenediamine derivative(s) of formula (I) and/or the addition salt(s) thereof with an acid represent from 0.0001% to 20% by weight relative to the total weight of the composition.

9. The composition as claimed in claim 8, characterized in that the substituted para-phenylenediamine derivative(s) of formula (I) and/or the addition salt(s) thereof with an acid represent from 0.001% to 15% by weight relative to the total weight of the composition.

10. The composition as claimed in claim 9, characterized in that the substituted para-phenylenediamine derivative(s) of formula (I) and/or the addition salt(s) thereof with an acid represent from 0.01% to 10% by weight relative to the total weight of the composition.

11. The composition as claimed in any one of the preceding claims, characterized in that the cationic polymers containing repeating units of formula (II) are chosen from those consisting of repeating units of formula (IV) below: 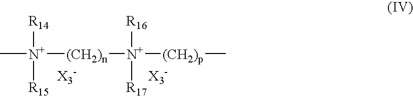
in which R₁₄, R₁₅, R₁₆ and R₁₇, which may be identical or different, denote an alkyl or hydroxyalkyl radical containing from 1 to 4 carbon atoms, n and p are integers ranging from 2 to 20 and X₃^{—} is an anion derived from a mineral or organic acid.

12. The composition as claimed in claim 11, characterized in that, in formula (IV), R₁₄, R₁₅, R₁₆ and R₁₇ denote the methyl radical, n=6 and p=3.

13. The composition as claimed in any one of claims 1 to 10, characterized in that the cationic polymers containing repeating units of formula (III) are chosen from those of formula (V) below:
—[—N⁺(CH₃)₂—(CH₂)ᵣ—NH—CO-D-NH—(CH₂)ᵣ—N⁺(CH₃)₂—(CH₂)₂)₂—O—(CH₂)₂—]—2X⁻ (V)
in which r denotes an integer ranging from 1 to 6, D is zero or represents a group —(CH₂)_{q}—CO— in which q denotes a number equal to 4 or to 7, and X⁻ is an anion derived from a mineral or organic acid.

14. The composition as claimed in claim 13, characterized in that, in formula (V), D is zero and X denotes a chlorine atom.

15. The composition as claimed in any one of the preceding claims, characterized in that the cationic polymer(s) containing repeating units of formula (III) represent(s) from 0.01% to 10% by weight relative to the total weight of the composition, preferably from 0.05% to 5% and even more preferentially from 0.1% to 3%.

16. The composition as claimed in any one of claims 1 to 10, characterized in that the amphoteric polymers are chosen from those resulting from the copolymerization of acrylic acid, or of methacrylic acid, or of maleic acid, or of α-chloroacrylic acid, with dimethyldiallylammonium chloride or with a dialkylaminoalkyl (meth)acrylate or a dialkylaminoalkyl (meth)acrylamide.

17. The composition as claimed in any one of claims 1-10 and 16, characterized in that the amphoteric polymer(s) represent(s) from 0.01% to 10% by weight, preferably from 0.05% to 5% by weight and more particularly from 0.1% to 3% by weight, relative to the total weight of the composition.

18. The composition as claimed in any one of claims 1 to 10, characterized in that the amphiphilic polymer(s) comprising at least one fatty chain is (are) of nonionic, anionic or cationic type.

19. The composition as claimed in claim 18, characterized in that the anionic amphiphilic polymers comprising at least one fatty chain are chosen from those comprising at least one (meth)acrylic acid unit and at least one fatty-chain allyl ether unit.

20. The composition as claimed in claim 18, characterized in that the anionic amphiphilic polymers comprising at least one fatty chain are chosen from those comprising at least one (meth)acrylic acid or ethacrylic acid unit, and at least one ester unit of C₁₀-C₃₀ alkyl and of unsaturated carboxylic acid.

21. The composition as claimed in claim 18, characterized in that the anionic amphiphilic polymers comprising at least one fatty chain are chosen from those comprising, among their monomers, an α,β-monoethylenically unsaturated carboxylic acid, an α,β-monoethylenically unsaturated carboxylic acid ester and an oxyalkylenated C₈-C₃₀ fatty alcohol.

22. The composition as claimed in claim 18, characterized in that the nonionic amphiphilic polymers comprising at least one fatty chain are chosen from celluloses modified with groups comprising at least one fatty chain, or hydroxypropylguars modified with groups comprising at least one fatty chain.

23. The composition as claimed in claim 18, characterized in that the nonionic amphiphilic polymers comprising at least one fatty chain are chosen from polyurethane polyethers comprising in their chain hydrophilic blocks of polyoxyethylenated nature and hydrophobic blocks that are aliphatic sequences alone and/or cycloaliphatic and/or aromatic sequences.

24. The composition as claimed in claim 18, characterized in that the nonionic amphiphilic polymers comprising at least one fatty chain are chosen from polymers with an aminoplast ether skeleton containing at least one fatty chain.

25. The composition as claimed in claim 18, characterized in that the cationic amphiphilic polymers comprising at least one fatty chain are chosen from quaternized cellulose derivatives and polyacrylates containing noncyclic amino side groups.

26. The composition as claimed in any one of claims 1-10 and 18-25, characterized in that the fatty-chain amphiphilic polymer(s) represent(s) from 0.01% to 10% and preferably from 0.1% to 5% by weight relative to the total weight of the composition.

27. The composition as claimed in any one of the preceding claims, characterized in that it contains at least one coupler.

28. The composition as claimed in claim 27, characterized in that the coupler(s) is (are) chosen from meta-phenylenediamines, meta-aminophenols, meta-diphenols and heterocyclic couplers, and the addition salts thereof with an acid.

29. The composition as claimed in claim 27 or 28, characterized in that the coupler(s) is (are) chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one and 1-phenyl-3-methylpyrazol-5-one, and the addition salts thereof with an acid.

30. The composition as claimed in any one of claims 27 to 29, characterized in that the coupler(s) is (are) present in a concentration of between 0.0001% and 15% by weight relative to the total weight of the composition.

31. The composition as claimed in any one of the preceding claims, characterized in that it contains at least one additional oxidation base and/or at least one direct dye.

32. The composition as claimed in claim 31, characterized in that the additional oxidation base(s) is (are) chosen from para-phenylenediamine, para-tolylenediamine, 2-hydroxyethyl-para-phenylenediamine, 1-N,N-bis(2-hydroxyethyl)-para-phenylenediamine, para-minophenols, ortho-phenylenediamines, ortho-aminophenols, double bases and heterocyclic bases.

33. The composition as claimed in claims 31 and 32, characterized in that the additional oxidation base(s) is (are) present in a concentration of between 0.0001% and 15% by weight relative to the total weight of the composition.

34. The composition as claimed in any one of the preceding claims, characterized in that the medium that is suitable for dyeing is an aqueous medium or a mixture of water and organic solvent.

35. The composition as claimed in claim 34, characterized in that the solvents are present in proportions of between 1% and 40% by weight relative to the total weight of the composition, and preferably between 5% and 30%.

36. The composition as claimed in any one of the preceding claims, characterized in that it contains sequestering agents, UV screening agents, waxes, volatile or nonvolatile, cyclic or linear or branched silicones, that may be organomodified or unmodified, preserving agents, ceramides, pseudoceramides, plant, mineral or synthetic oils, vitamins or provitamins, opacifiers, thickeners, reducing agents or antioxidants, fatty alcohols and acidifying or basifying agents.

37. The composition as claimed in any one of the preceding claims, characterized in that it contains at least one anionic, nonionic, cationic or amphoteric surfactant and preferably a nonionic surfactant.

38. A ready-to-use composition for the oxidation dyeing of keratin fibers, in particular of human keratin fibers such as the hair, characterized in that it is obtained by mixing a composition as defined in any one of claims 1 to 37 and a composition containing at least one oxidizing agent.

39. The composition as claimed in claim 38, characterized in that the pH is between the values 4 and 12 and preferably between 6 and 11.

40. The composition as claimed in claim 38, characterized in that the oxidizing agent is chosen from hydrogen peroxide, urea peroxide, alkali metal bromates or ferricyanides, persalts such as perborates and persulfates, and redox enzymes, where appropriate in the presence of their respective donor or cofactor.

41. A composition as claimed in claim 40, characterized in that the oxidizing agent is an aqueous hydrogen peroxide solution with a titer ranging from 1 to 40 volumes and preferably 5 to 40 volumes.

42. A process for the oxidation dyeing of keratin fibers, and in particular of human keratin fibers such as the hair, which consists in applying to the fibers a dye composition containing, in a medium that is suitable for dyeing, at least one substituted para-phenylenediamine derivative of formula (I) defined in any one of claims 1-10, in combination with at least one polymer chosen from amphoteric polymers, cationic polymers containing repeating units of formula (II) or (III), or amphiphilic polymers comprising at least one fatty chain and defined in any one of claims 11 to 26, the color being developed at alkaline, neutral or acidic pH with the aid of a composition containing at least one oxidizing agent, which is mixed just at the time of use to the dye composition, or which is applied sequentially without intermediate rinsing.

43. The process as claimed in claim 42, characterized in that it consists in applying the ready-to-use composition, prepared extemporaneously at the time of use from the dye composition according to the invention and the oxidizing composition, to the wet or dry keratin fibers, and leaving it to act for an exposure time preferably ranging from 1 to 60 minutes approximately and more preferably from 10 to 45 minutes approximately, rinsing the fibers and then optionally washing them with shampoo, followed by rinsing them again and drying them.

44. A multi-compartment device or “kit” for the oxidation dyeing of keratin fibers, in particular human keratin fibers such as the hair, characterized in that it comprises at least one compartment containing at least one substituted para-phenylenediamine derivative of formula (I) defined in any one of claims 1-10 and at least one polymer chosen from amphoteric polymers, cationic polymers containing repeating units of formula (II) or (III), or amphiphilic polymers comprising at least one fatty chain and defined in any one of claims 11 to 26, and at least one other compartment containing at least one oxidizing agent.

45. A multi-compartment device or “kit” for the oxidation dyeing of keratin fibers, in particular human keratin fibers such as the hair, characterized in that it comprises at least one compartment containing at least one substituted para-phenylenediamine derivative of formula (I) defined in any one of claims 1-10, optionally in combination with at least one polymer chosen from amphoteric polymers, cationic polymers containing repeating units of formula (II) or (III), or amphiphilic polymers comprising at least one fatty chain and defined in any one of claims 11-26, and another compartment containing an oxidizing agent in combination with at least one polymer chosen from amphoteric polymers, cationic polymers containing repeating units of formula (II) or (III), or amphiphilic polymers comprising at least one fatty chain and defined in any one of claims 7-22.

46. A multi-compartment device or “kit” for the oxidation dyeing of keratin fibers, in particular of human keratin fibers such as the hair, characterized in that it comprises at least one compartment containing at least one substituted para-phenylenediamine derivative of formula (I) defined in any one of claims 1-10, at least one other compartment containing at least one polymer chosen from amphoteric polymers, cationic polymers containing repeating units of formula (II) or (III), or amphiphilic polymers comprising at least one fatty chain and defined in any one of claims 11-26, and at least one other compartment containing an oxidizing agent.
